(19) United States
(12) Patent Application Publication
(10) Pub. No.: US 2005/0283237 A1
(43) Pub. Date: Dec. 22, 2005

(54) Artificial spinal disk replacement device with staggered vertebral body attachments

(75) Inventors: James F. Zucherman, San Francisco, CA (US); Ken Y. Hsu, San Francisco, CA (US); Charles J. Winslow, Walnut Creek, CA (US); Henry A. Klyce, Piedmont, CA (US)

Correspondence Address: FLIESLER MEYER, LLP, FOUR EMBARCADERO CENTER, SUITE 400, SAN FRANCISCO, CA, 94111, US

(73) Assignee: St. Francis Medical Technologies, Inc., Alameda, CA (US)

(21) Appl. No.: 10/994,595

(22) Filed: Nov. 22, 2004

Related U.S. Application Data
(60) Provisional application No. 60/524,463, filed on Nov. 24, 2003.

(51) Int. Cl. A61F 2/44

(52) U.S. Cl. 623/17.11; 623/17.14

(57) ABSTRACT

An intervertebral disk implant is described that has flanges designed to maximize mechanical strength, and at the same time is designed to provide for spatial complementarity of the flanges. In this regard, multiple devices can be implanted between consecutive intervertebral spaces, since the spatially complementary configuration of the flanges allow more than one device to be securely and conveniently anchored on the body of the same vertebral body.

### CLAIM OF PRIORITY

This application claims priority under 35 USC 119 to U.S. Provisional Patent Application No. 60/524,463, filed Nov. 24, 2003, entitled “ARTIFICIAL VERTEBRAL DISK REPLACEMENT IMPLANT WITH STAGGERED VERTEBRAL BODY ATTACHMENTS,” which is incorporated herein by reference.

### CROSS-REFERENCE TO RELATED APPLICATIONS

This application is related to U.S. Provisional Application No. 60/422,039, filed on Oct. 29, 2002, entitled “ARTIFICIAL VERTEBRAL DISK REPLACEMENT IMPLANT WITH TRANSLATING PIVOT POINT AND METHOD,” U.S. patent application Ser. No. 10/684,669, filed Oct. 14, 2003, entitled “ARTIFICIAL VERTEBRAL DISK REPLACEMENT IMPLANT WITH TRANSLATING PIVOT POINT AND METHOD,” U.S. patent application Ser. No. 10/684,668, filed Oct. 14, 2003, entitled “ARTIFICIAL VERTEBRAL DISK REPLACEMENT IMPLANT WITH CROSSBAR SPACER AND METHOD,” U.S. Provisional Application No. 60/517,973, filed on Nov. 6, 2003, entitled “ARTIFICIAL VERTEBRAL DISK REPLACEMENT IMPLANT WITH CROSSBAR SPACER AND LATERAL IMPLANT METHOD,” U.S. Provisional Application No. 60/422,022, filed October 29, 2002, entitled “ARTIFICIAL VERTEBRAL DISK REPLACEMENT IMPLANT WITH A SPACER AND METHOD,” and U.S. patent application Ser. No. 10/685,011, filed Oct. 14, 2003, entitled “ARTIFICIAL VERTEBRAL DISK REPLACEMENT IMPLANT WITH SPACER AND METHOD,” all of which are incorporated herein by reference.

### BACKGROUND

The field of art of this disclosure is a device and method for replacement of intervertebral disks.

The spinal column is a biomechanical structure composed primarily of ligaments, muscles, vertebrae and intervertebral disks. The biomechanical functions of the spine include: (1) support of the body, which involves the transfer of the weight and the bending movements of the head, trunk and arms to the pelvis and legs, (2) complex physiological motion between these parts, and (3) protection of the spinal cord and the nerve roots.

As the present society ages, it is anticipated that there will be an increase in adverse spinal conditions which are characteristic of aging. By way of example, with aging comes an increase in spinal stenosis (including, but not limited to, central canal and lateral stenosis), and facet arthroplasty. Spinal stenosis typically results from the thickening of the bones that make up the spinal column and is characterized by a reduction in the available space for the passage of blood vessels and nerves. Pain associated with such stenosis can be relieved by medication and/or surgery.

In addition to spinal stenosis, and facet arthroplasty, the incidence of damage to the intervertebral disks is also common. The primary purpose of the intervertebral disk is to act as a shock absorber. The disk is constructed of an inner gel-like structure, the nucleus pulposus (the nucleus), and an outer rigid structure comprised of collagen fibers, the annulus fibrosus (the annulus). At birth, the disk is 80% water, and then gradually diminishes, becoming stiff, With age, disks may degenerate, and bulge, thin, herniate, or ossify. Additionally, damage to disks may occur as a result trauma or injury to the spine.

The damage to disks may call for a range of restorative procedures. If the damage is not extensive, repair may be indicated, while extensive damage may indicate full replacement. Regarding the evolution of restoration of damage to intervertebral disks, rigid fixation procedures resulting in fusion are still the most commonly performed surgical intervention. However, trends suggest a move away from such procedures. Currently, areas evolving to address the shortcomings of fusion for remediation of disk damage include technologies and procedures that preserve or repair the annulus, that replace or repair the nucleus, and that utilize technology advancement on devices for total disk replacement. The trend away from fusion is driven both by issues concerning the quality of life for those suffering from damaged intervertebral disks, as well as responsible health care management. These issues drive the desire for procedures that can be tolerated by patients of all ages, especially seniors, and can be performed preferably on an outpatient basis.

Most recently, there has been an increased interest in replacing dysfunctional disks with artificial disks instead of fusing together adjacent vertebral bodies. A number of artificial disks are beginning to appear in the medical marketplace, which vary greatly in shape, design and functionality. One current challenge for artificial disk replacement devices concerns anchoring the devices to the limited surface of the vertebral bodies. Generally, these devices include fixation devices, principally screws that are closely positioned on the anterior surface of the vertebral body. Due to factors such as the limited space on and the quality of the bone of the vertebral bodies, there is a need to optimally select the placement of the screws so that maximum fixation can be obtained.

Accordingly, there is a need in the art for innovation in technologies and methods that advance the art in the area of intervertebral disk replacement. This not only enhances the quality of life for those suffering from the condition, but is responsive to the current needs of health care management.

### BRIEF DESCRIPTION OF THE DRAWINGS

FIGS. 1A-1C represent one embodiment of the disclosed intervertebral device: FIG. 1A is a front view of one embodiment. FIG. 1B is a side view of the embodiment of FIG. 1A. FIG. 1C shows two devices implanted in consecutive vertebrae, and depicts the interdigitating nature of the flanges of the embodiment of FIG. 1A.

FIGS. 2A-2C represent a second embodiment of the disclosed intervertebral device: FIG. 2A is a front view of the second embodiment. FIG. 2B is a side view of the embodiment of FIG. 2A. FIG. 2C shows two devices implanted in consecutive vertebrae, and depicts the interdigitating nature of the flanges of the embodiment of FIG. 2A.

FIGS. 3A-3B show prior art devices where the flanges are not interdigitating.

### DETAILED DESCRIPTION OF THE PRESENT INVENTION

What is disclosed is an intervertebral implantation device designed to allow the natural movement of the spine; axial rotation, lateral bending, forward flexion, and backward extension. The design of the device includes flanges that are spatially complementary for anchoring the device to vertebrae, allowing multiple devices to be inserted in consecutive vertebrae so that the flanges are interdigitated. The device can be fabricated from a variety of materials, as well as being a composite of materials.

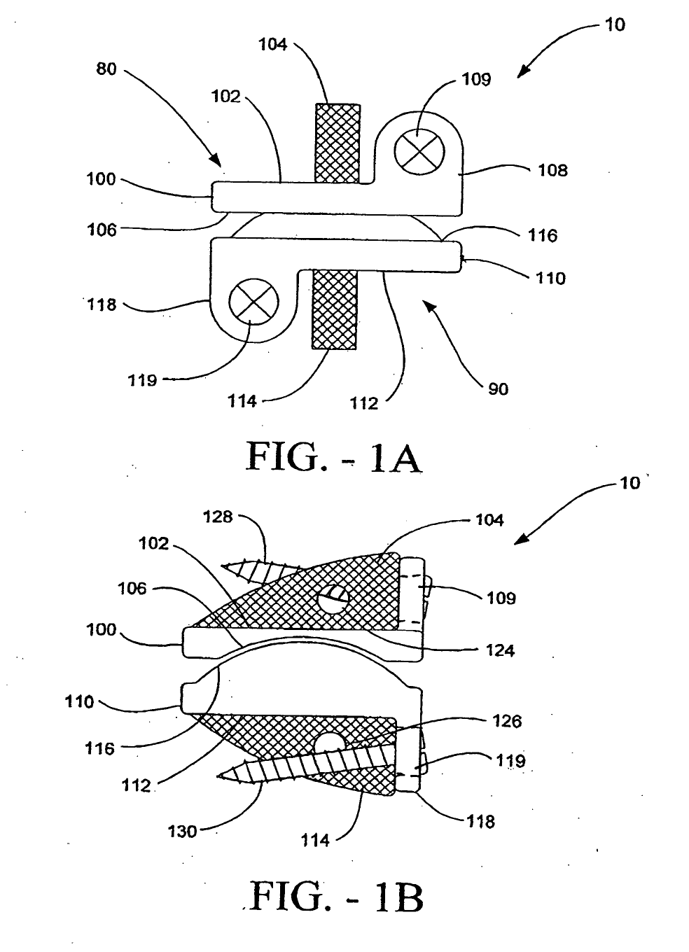

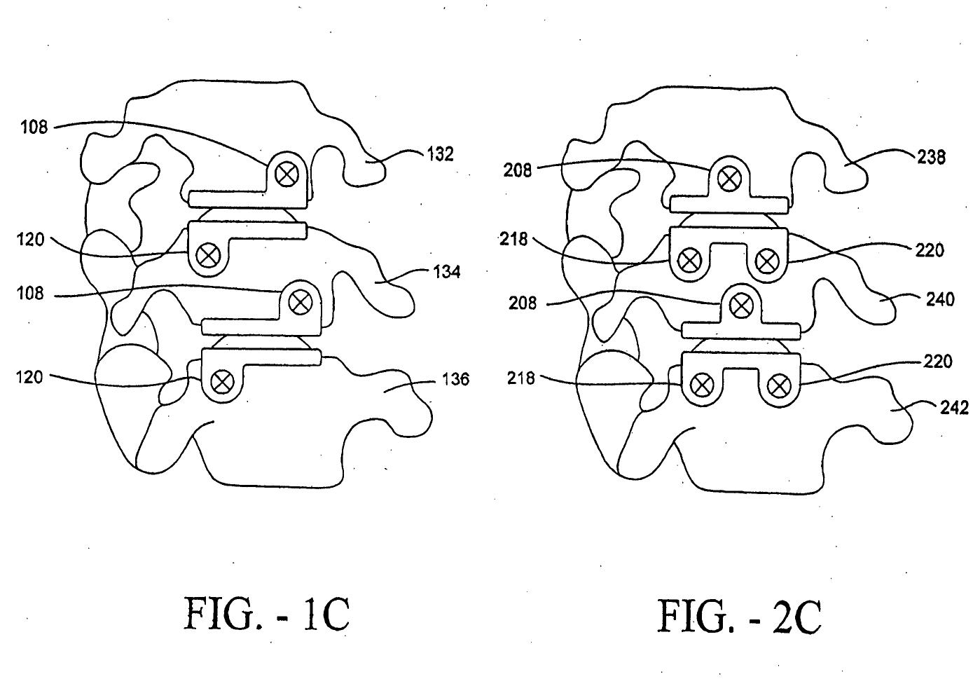

FIGS. 1A-1C show one embodiment 10 of the device, having an upper part or end plate 80 and a lower part or end plate 90. The first end plate 100 has a first outer surface 102 having a first keel 104, a first inner surface 106, and a first or upper flange 108 having a first through-hole 109. Similarly, the second end plate 110 has a second outer surface 112 having second keel 114, a second inner surface 116, and a second or lower flange 118 having a second through-hole 119. The inner surface 116 of the second end plate 110 of FIG. 1A is shown to be raised and hemispherical. In FIG. 1B, a side view of the device is shown. In side view of this embodiment of implant 10, the second inner surface 116 of the second end plate 110 of the lower part 90 serves as a spacer and is convex as well as hemispherical. The spacer is preferably matched to the concave and hemispherical first inner surface 106 of the first end plate 100 of the upper part 80 to form the overall shape of the spacer. The design of these matching first and second inner surfaces 106,116 of the spacer, as shown in FIGS. 1A-1B, facilitates the natural movement associated with a healthy disk when the device is implanted. It is contemplated that the spacer alternatively has a crossbar spacer configuration, a curved spacer configuration or an elongated spacer configuration. More details regarding these alternative designs are discussed in U.S. patent applications Ser. No. 10/684,668; 10/684,669; and 10/685,011, all of which are incorporated by reference above.

Further, first outer surface 102, and second outer surface 112 have features that facilitate bone in-growth, so that the device can become mechanically stabilized within the intervertebral space over time. FIG. 1C shows two devices implanted into the intervertebral spaces of consecutive vertebra, demonstrating the staggered nature of the first and second flanges 108,118.

As is evident from FIG. 1A, the upper flange 108 of the upper part 80 and the lower flange 118 of the lower part 90 of the device 10 are not aligned but are in a staggered configuration. In the embodiment depicted in FIG. 1A, the upper flange 108 is shown disposed to the right end of the implant body and in this particular embodiment to the right side of the first keel 104. The lower flange 120 is disposed to the left end of the implant body and in this particular embodiment, to the left side of the keel 114. As is evident from FIG. 1C, the staggered configuration allows for maximum spacing between the placement of the screw 130 of the lower part 90 of a first device 10 which is placed in a vertebral body and the screw 128 of the upper part 80 of a second device 10 which is placed in the same vertebral body. It is to be understood that the bone that comprises the vertebral body is porous, and with greater spacing, the screws can have maximum fixation to the vertebral body.

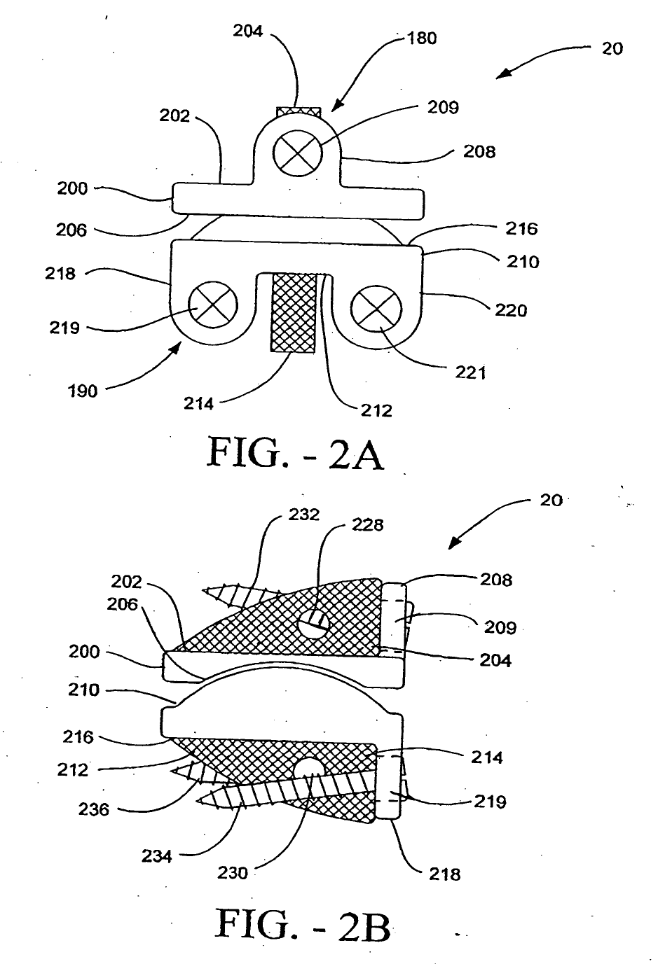

Another embodiment of the disclosed device is shown in FIGS. 2A-2C. This embodiment is similarly characterized by the first end plate 200 having a first outer surface 202 with a first keel 204, a first inner surface 206 and a first or upper flange 208 having a first through-hole 209. In this embodiment, the second end plate 210 has a second outer surface 212 having a second keel 214 and a second inner surface 216. The embodiment is shown having a pair of lower flanges, or second and third flange, 218, 220 with through-holes 219, 221, respectively. The embodiment of the intervertebral device 20 in FIGS. 2A-2B has first and second inner surfaces 206,216 that facilitate the natural movement associated with a healthy disk when the device is implanted. Additionally, the first outer surface 202, and the second outer surface 212 have features that facilitate bone ingrowth for promoting mechanical stability of the implanted device, which will be subsequently discussed in more detail. FIG. 2C shows two devices implanted into the intervertebral spaces of consecutive vertebra, demonstrating the staggered configuration of the first flange 208 with the second and third flanges 118,220.

As is evident from FIG. 2A, the upper flange 208 of the upper part 180 and the lower flanges 218,220 of the lower part 190 of the device 20 are not aligned, but are in a staggered configuration. In the embodiment depicted, the upper flange 208 is disposed in the center of the implant body at a mid-point and in-line with the first keel 204. As shown in FIGS. 2A-2B, the keel 204 is shown to include an aperture therethrough which is preferably in-line and aligned with the through-hole 209 in the upper flange 208. As shown in FIGS. 2A-2B, the aperture in the keel 204 accepts the screw 232 to provide a secure attachment of the upper end plate 100 to the upper vertebral body. The lower flanges 218,220 are disposed to the right and left of the implant body of the lower part 190 and in particular are disposed to the right and the left of the second keel 214 respectively. As is evident from FIG. 2C, this staggered configuration allows for maximum spacing between the placement of the screw 209 of an upper part 180 of a first device 20 which is placed in a vertebral body and the right and left screws 219,221 of a lower part 190 of a second device 20 which is placed in the same vertebral body. Again it is to be understood that the bone that comprises the vertebral body is porous and thus with greater spacing the screws can have maximum fixation to the vertebral body.

The keels 104, 114 are oriented to protrude from the outer surfaces 102, 112 of the upper and lower end plates, respectively. In one embodiment, the keels 104, 114 are oriented lengthwise to extend between the anterior and posterior sides of the end plates. In another embodiment, the keels 104, 114 are oriented lengthwise to extend between the lateral sides of the end plates (i.e. perpendicular to the sagittal plane of the patient's spine).

In another embodiment, the keel and the plate can be a fabricated as single part, or in yet another embodiment as multiple pieces assembled as an intact part. Materials contemplated for use in the device fabrication have enough strength to withstand the continuous wear at the inner surfaces, and yet are suitable to serve the function of absorbing shock. To ensure long-term mechanical stability in the intervertebral space, materials are selected that have excellent properties for osseointegration. Osseointegration is the ability of a material to join with bone and other tissue. Additionally, materials are selected for their biocompatibility, which means that a material causes no untoward effect to the host; e.g. chronic inflammation, thrombosis, and the like.

Medical grade stainless steel alloys and cobalt chrome are well known materials as candidates for medical implants that are load-bearing. One material considered to rank highly across a number of desirable attributes such as strength, biocompatibility, and osseointegration is medical grade titanium, and alloys thereof.

The outer surfaces of the device shown in FIGS. 1A-1C and 2A-2C are configured to have surface roughening, since surface texturing of implants is known to facilitate bone ingrowth. In addition to the choice of material, and surface roughening of the device, in FIGS. 1C and 2C the holes 124, 126 and 228, 230, respectively, are also features in the outer surface for the facilitation of bone ingrowth.

In another embodiment of the disclosed device, the keels 100, 112, 200, 212 and the plates 104,116, 204, 216 can be made of different materials. For example, the plate can be fabricated from titanium, or alloys thereof, while the keel can be fabricated from polymeric materials.

Interesting classes of polymers are biocompatible polymers. Copolymers, blends, and composites of polymers are also contemplated for fabrication in the disclosed device. A copolymer is a polymer derived from more than one species of monomer. A polymer composite is a heterogeneous combination of two or more materials, wherein the constituents are not miscible, and therefore exhibit an interface between one another. A polymer blend is a macroscopically homogeneous mixture of two or more different species of polymer.

To reinforce a polymeric material, fillers, are added to a polymer, copolymer, polymer blend, or polymer composite. Fillers are added to modify properties, such as mechanical, optical, and thermal properties. In this case, fillers, such as carbon fibers, are added to reinforce the polymers mechanically to enhance strength for certain uses, such as load bearing devices.

One group of biocompatible polymers are the polyaryletherketones which has several members, which include polyetheretherketone (PEEK), and polyetherketoneketone (PEKK). PEEK has proven as a durable material for implants, as well as meeting criteria of biocompatibility. Medical grade PEEK is available from Victrex Corporation under the product name PEEK-OPTIMA. Medical grade PEKK is available from Oxford Performance Materials under the name OXPEKK, and also from CoorsTek under the name BioPEKK. These medical grade materials are also available as reinforced polymer resins, such reinforced resins displaying even greater material strength.

As will be appreciated by those of skill in the art, materials of different types can be used to fabricate the device. For instance, the keels 100, 112, 200, 212 and the plates 104, 116, 204, 216 can be made of titanium, and the outer surfaces 102, 114, 202, 214 of the keels 100, 112, 200, 212, or the plates 104,116, 204, 216 coated with a thin film of a biocompatible material. In another embodiment, it is contemplated that the keels 100, 112, 200, 212 can be fabricated from a polymeric material, while the plates 104,116, 204, 216 can be fabricated from titanium. In still another embodiment contemplated, the keels 100, 112, 200, 212 are a combination of a polyaryletherketone, such as PEKK®, with a thin layer of a bioresorbable polymer, or polymer composite used for the fabrication of the outer surfaces 102, 114, 202, 214.

Initially, if not permanently, the implanted device can be stabilized in the intervertebral space by using fasteners to secure the device to the body of a vertebrae. Depending on region of the spine, the device can require only temporary stabilization until bone ingrowth occurs. In that case, the use of biodegradable fasteners can be desirable.

One type of fastener that can be used is a biodegradable pedicle screw. The time to total resorption varies for different kinds of biodegradable polymer. Biodegradable screws can have total time to resorption from 6 months to 5 years. Biologically Quite (Instrument Makar), a poly(D,L-lactide-co-glycolide) screw degrades in ca. 6 months, while Phusiline (Phusis), a poly(L-lactide-co-D,L lactide) copolymer degrades in ca. 5 years, and Bioscrew (Linvatec), a ploy(L-lactide) screw degrades in the range of 2-3 years.

If permanent anchoring is desirable, pedicle screws of medical grade titanium and alloys thereof are available from a number of manufacturers, such as Acromed, Medtronic, Instratek, and Stryker. Alternatively, polymeric pedicle screws from the polyarylketone family, such as PEKK, are also available. Pedicles screws from made PEKK resin known as OXPEKK® are available from Oxford Performance Materials, and have excellent mechanical properties, and proven track record of biocompatibility.

Again, the flanges 108, 118 of device 10 or flanges 208, 218, 220 of device 20 are designed for maximum mechanical stability at the site of device anchoring, while at the same time conserving space by being spatially complementary. The flange design of the disclosed device allows for multiple devices to be implanted in consecutive vertebrae, with the flanges of more than one device anchored on the body of a single vertebrae. For vertebrae that are more closely spaced, such as cervical vertebrae, this can be desirable.

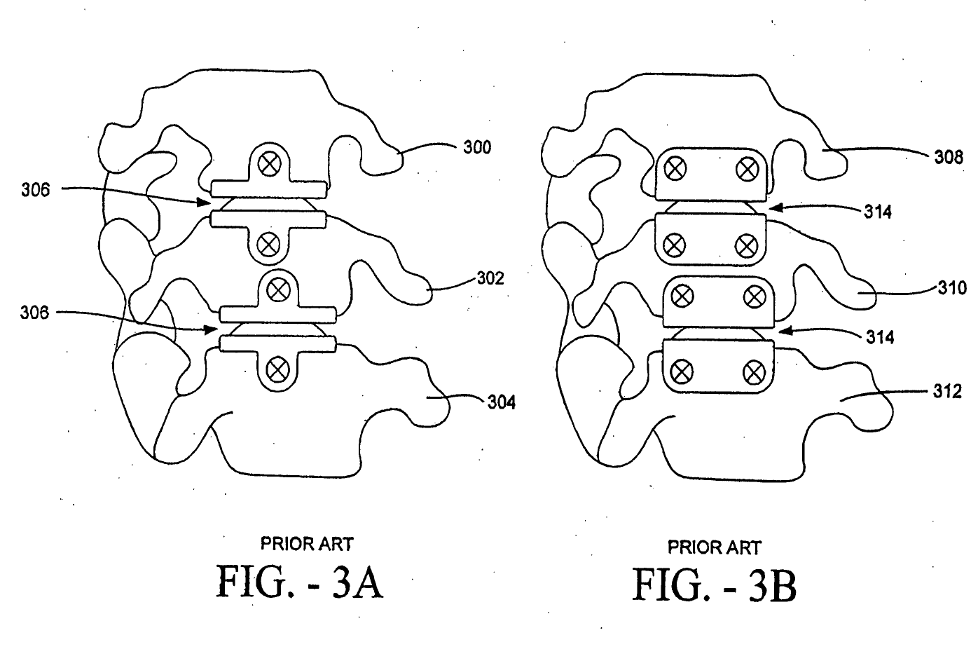

The manner in which the design maximizes mechanical stability, while conserving space is readily understood by referring to FIGS. 1C and 2C in comparison to FIGS. 3A-3B. In these figures, devices 10 and 20 are shown implanted between vertebrae 132,134,136 and 238, 240,242 respectively. It is evident that the design of the devices shown in FIGS. 1C and 2C allows for an interdigitating arrangement of the flanges of two or more devices implanted between consecutive vertebrae. This is in contrast to the flange design of prior art devices 302, 308 shown in FIGS. 3A-3B inserted between consecutive vertebrae 300, 304, and 306,310 respectively. Here, due to the lack of spatial complementarity of the flanges of consecutive devices, the ready implantation of multiple devices between consecutive vertebrae may be contraindicated.

What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

1. An intervertebral artificial disk implant device comprising:
a. a first end plate having a first keel protruding from a first outer surface, the first end plate including a first flange extending therefrom along a side; and
b. a second end plate having a second keel protruding from a second outer surface, the second end plate including a second flange extending therefrom along the side.

2. The implant of claim 1 wherein the first and second keels are positioned substantially at a midpoint with respect to the side.

3. The implant of claim 1 wherein the first flange is positioned at a midpoint along the side.

4. The implant of claim 3 wherein the first keel further comprises an aperture therethrough, wherein the aperture is aligned with an aperture through the first flange.

5. The implant of claim 3 wherein the second flange is positioned between the midpoint and an end of the second end plate.

6. The implant of claim 3 further comprising a third flange positioned between the midpoint and an end of the second end plate.

7. The implant of claim 1 wherein the first flange is positioned between a midpoint and a first end along the side.

8. The implant of claim 7 wherein the second flange is positioned between the midpoint and a second end opposite of the first end along the side.

9. The implant of claim 1 wherein first and second end plates are configured to promote bone ingrowth.

10. The implant of claim 9 wherein an outer surface of the first end plate and the second end plate is at least partially textured.

11. The implant of claim 9 wherein an outer surface of the first end plate and the second end plate includes at least one aperture therethrough.

12. The implant of claim 1 wherein the first and second end plates are made of at least one biocompatible material.

13. The implant of claim 12 wherein the biocompatible material is a biocompatible metal.

14. The implant of claim 12 wherein the biocompatible metal is stainless steel.

15. The implant of claim 12 wherein the biocompatible metal is titanium.

16. The implant of claim 12 wherein the biocompatible material is a polymer.

17. The implant of claim 16 wherein the polymer is a polyarylesterketone.

18. The implant of claim 17 wherein the polyarylesterketone is reinforced.

19. An intervertebral implant comprising:
a. a first end plate having a first keel extending from a first outer surface and having a first flange substantially perpendicular to the first outer surface; and
b. a second end plate having a second keel extending from a second outer surface and having a second flange substantially perpendicular to the second outer surface, wherein the first and second flanges are spatially complementary.

20. The implant of claim 19 wherein the first flange and the second flange are arranged in a staggered configuration when the implant is inserted between adjacent vertebral bodies.

21. The implant of claim 19 wherein the first flange is adjacent to the first keel and the second flange is adjacent to the second keel.

22. The implant of claim 19 wherein the first flange is adjacent to the first keel and the second flange is adjacent to the second keel, wherein the first and second keels extend between an anterior end and a posterior end of the respective end plates.

23. The implant of claim 19 wherein the first flange is adjacent to the first keel and the second flange is adjacent to the second keel, wherein the first and second keels extend between lateral ends of the respective end plates.

24. The implant of claim 19 wherein the first flange is in-line with the first keel.

25. The implant of claim 24 wherein the second end plate further comprises a third flange, wherein the second flange and the third flange are adjacent to the second keel.

26. The implant of claim 19 wherein the first flange is in-line with the first keel, wherein the first keel includes an aperture aligned with a first aperture in the first flange.

27. The implant of claim 19 wherein first and second end plates are configured to promote bone ingrowth.

28. An intervertebral implant comprising:
a. an articulating unit having an upper flange and a lower flange located on a same side of the articulating unit, the upper and lower flanges located proximal to opposing ends of the articulating unit; and
b. a spacer positioned within the articulating unit.

29. The implant of claim 28 wherein the articulating unit further comprises:
a. a first end plate having a first keel extending from a first outer surface; and
b. a second end plate having a second keel extending from a second outer surface.

30. The implant of claim 29 wherein the first keel extends between an anterior end and a posterior end of the first end plate.

31. The implant of claim 29 wherein the second keel extends between an anterior end and a posterior end of the second end plate.

32. The implant of claim 29 wherein the first keel extends between a first lateral side and a second lateral side of the first end plate.

33. The implant of claim 29 wherein the second keel extends between a first lateral side and a second lateral side of the second end plate.

34. An intervertebral implant comprising:
a. a first end plate having a first keel extending from a first outer surface and having a first flange in-line with the first keel; and
b. a second end plate having a second keel extending from a second outer surface, the second end plate having a second flange and a third flange adjacent to the second keel.

35. An intervertebral implant comprising:
a. a first end plate having a first keel extending from a first outer surface and having a first flange in-line with the first keel, the first keel having an aperture therethrough aligned with an aperture in the first flange; and
b. a second end plate having a second keel extending from a second outer surface, the second end plate having a second flange and a third flange adjacent to the second keel.
